(19) United States
(12) Patent Application Publication
(10) Pub. No.: US 2008/0221387 A1
(43) Pub. Date: Sep. 11, 2008

(54) Vibrating toothbrush attachment

(76) Inventor: Cynthia L. Gaboury, Tallahassee, FL (US)

Correspondence Address: Pennington, Moore, Wilkinson, Bell & Dunbar, P.A., Post Office Box 10095, Tallahassee, FL, 32302-2095, US

(21) Appl. No.: 12/151,882

(22) Filed: May 9, 2008

Related U.S. Application Data
(63) Continuation-in-part of application No. 11/072,379, filed on Mar. 4, 2005.

(51) Int. Cl. A61F 5/00; A61H 1/00; A46B 13/00

(52) U.S. Cl. 600/38; 601/70; 601/46; 15/22.1

(57) ABSTRACT

An attachment comprised of a tip member, head and wand for attachment to an electric toothbrush for use as a sexual aid or muscular stimulation. The preferred embodiment of the attachment includes an oscillating tip member with knob-like protrusions, a head and a wand provided in a single, integrated unit for receiving a standard electric toothbrush.

### MICROFICHE APPENDIX

Not Applicable

### BACKGROUND OF THE INVENTION

1. Field of the Invention

This invention relates to the field of vibrating devices used for sexual or muscular stimulation. More specifically, the invention comprises a molded shaft which can be attached to an electric toothbrush.

2. Description of the Related Art

The use of sexual aids such as vibrators of various designs and configurations is known in the prior art. These devices are typically phallic in shape (although unconventional shapes are also employed in the prior art) and are commonly used to stimulate the erogenous zones of the body including the external and internal genitalia. Some sexual aids employ a vibrating mechanism to increase the sexual stimulation provided by the device. These sexual aids are often referred to as vibrators.

It is often desirable for a vibrator to be transportable so that the user can bring the device with them when they travel. Since conventional vibrators employ internal batteries and a motor to create the vibrating motion of the device, the devices can be discovered during luggage screening at airports and in other places where luggage screening is employed. The discovery of a vibrator in these circumstances can create an uncomfortable and embarrassing situation for people who prefer to keep their use of sexual aids private.

### BRIEF SUMMARY OF THE INVENTION

The present invention overcomes the problems of the prior art by providing a vibrating device which is discreet and transportable. This vibrating device can be used for muscular or sexual stimulation. The invention comprises a molded plastic shaft assembly which is adapted to fit into an electric toothbrush. The shaft assembly features a cap with knob-like protrusions for enhanced stimulation.

In one embodiment the cap features a groove for secured attachment of the cap to the toothbrush.

Another embodiment provides for the use of an adhesive for permanent attachment of the cap to the toothbrush.

In yet another embodiment, a tip member is integrated with the wand and connects to the female coupling portion of the electric toothbrush.

The invention provides all of these features, advantages, and objects along with others that will become apparent with reference to the following description and accompanying drawings.

### BRIEF DESCRIPTION OF THE SEVERAL VIEWS OF THE DRAWINGS

FIG. 1 is a perspective view, showing an electric toothbrush.

FIG. 2 is a perspective view, showing the present invention.

FIG. 3 is a top view, showing the present invention.

FIG. 4 is a perspective view, showing the present invention.

FIG. 5 is a bottom view, showing an alternate embodiment of the present invention.

FIG. 6 is a perspective view, showing the present invention attached to an electric toothbrush.

FIG. 7 is a perspective view, showing an alternate embodiment of the present invention.

FIG. 8 is a perspective view, showing an alternate embodiment of the present invention attached to an electric toothbrush.

FIG. 9 is a perspective view, showing an alternate embodiment of the present invention.

FIG. 10 is a perspective view, showing the inside of the wand in the present invention.

FIG. 11 is a perspective view, showing a view of the cap portion of the present invention.

FIG. 12 is a back view, showing the cap portion of the present invention.

FIG. 13 is a front view, showing the one region of the present invention.

FIG. 14 is a perspective view, showing the component parts of the present invention.

FIG. 15 is a perspective view, showing the component parts of the present invention.

FIG. 16 is a perspective view, showing the assembly of the component parts of the present invention.

FIG. 17 is a perspective view, showing the prior art electric toothbrush handle.

FIG. 18 is a perspective view, showing the connection between the electric toothbrush handle and one embodiment of the present invention.

FIG. 19 is a perspective view, showing an alternate embodiment of the present invention.

FIG. 20 is a perspective view, showing another view of an alternate embodiment of the present invention.

FIG. 21 is a perspective view, showing the protrusions on an alternate embodiment of the present invention.

FIGS. 22 A and B are perspective views showing two views of a protrusion on an alternate embodiment of the present invention.

FIGS. 23 A, B, C and D are top views showing the motion of the movement dowel in relation to the slide.

REFERENCE NUMERALS IN THE DRAWINGS

| | | | |
|---|---|---|---|
| 10 | cap | 12 | top |
| 14 | knobs | 16 | bore |
| 18 | counterbore | 20 | compartments |
| 22 | ridges | 24 | lip |
| 26 | grip | 28 | wand |
| 30 | brush head | 32 | bristles |
| 34 | sidewall | 36 | bottom |
| 38 | electric toothbrush | 40 | knob-to-knob distance |
| 42 | brush-head seat | 44 | groove |
| 46 | shaft assembly | 48 | wand |
| 50 | male coupling portion | 52 | female coupling portion |
| 56 | head | 58 | transfer |
| 60 | rotating disc | 62 | support disc |
| 64 | support peg | 66 | stop |
| 68 | slide | 70 | flange |
| 72 | movement dowel | 74 | coupling |
| 76 | notch | 78 | deformable cap |
| 80 | back | 82 | edge |
| 84 | crown | 86 | fingers |
| 88 | ledge | 90 | tip member |
| 92 | sidewall | 94 | knob-like protrusions |
| 96 | top portion | | |

### DETAILED DESCRIPTION OF THE INVENTION

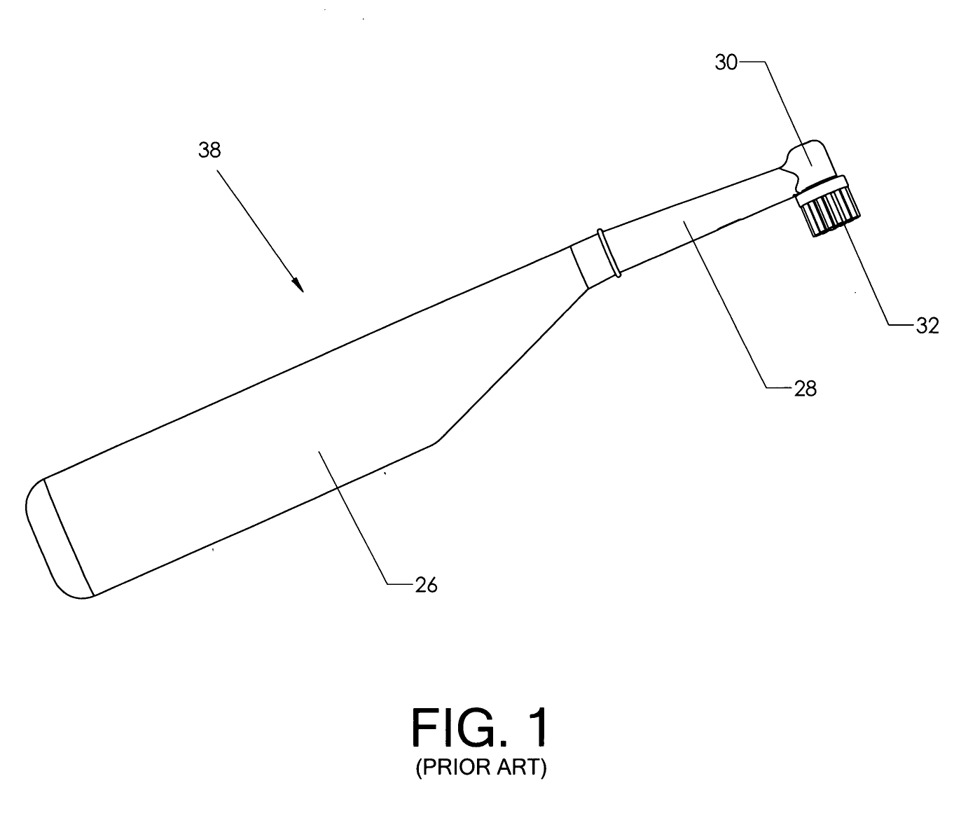

A conventional electric toothbrush is shown in FIG. 1. Electric toothbrush 38 is generally composed of grip 26 and wand 28. Aside from serving as means for holding the toothbrush while a user brushes their teeth, grip 26 also serves as a housing for an internal motor and power supply. Most electric toothbrushes are powered by replaceable batteries, but some have an internal rechargeable power source. Grip 26 also generally features an on/off switch which allows the user to actuate the internal motor.

Wand 28 is attached to grip 26. Wand 28 includes brush head 30 and bristles 32. In most electric toothbrushes, wand 28 and grip 26 are detachable, so that the user can replace wand 28 when bristles 32 become worn. Wand 28 also houses a transfer (not shown) that converts the motion created by the internal motor of electric toothbrush 38 into an oscillating motion in brush head 30. In most electric toothbrushes, the head does not spin only in one direction. Instead most brush heads of electric toothbrushes oscillate having alternating positive and negative angles of rotation. Conventional electric toothbrushes typically have a range of angular rotation from +/−20° to +/−100°.

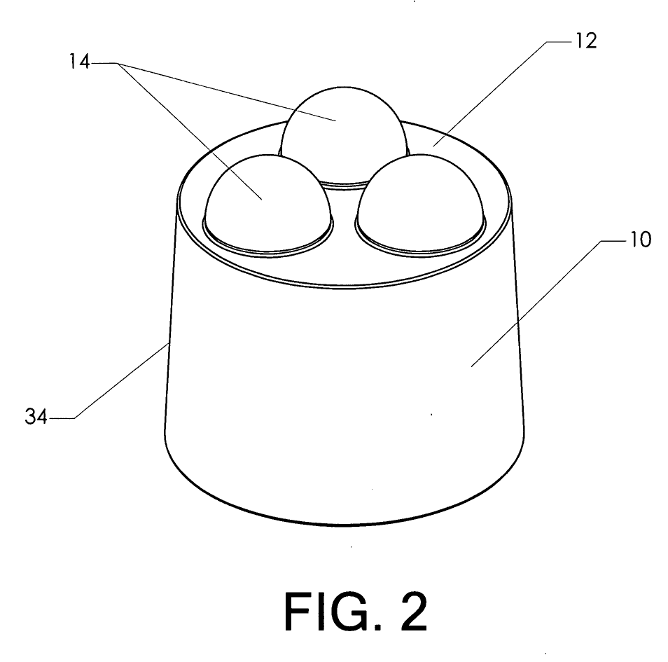

A vibrating cap for attachment to an electric toothbrush is shown in FIG. 2. Cap 10 has sidewall 34 and top 12. Cap 10 can be made of rubber, latex, plastic or other nonallergenic varieties of polymers by the process of injection molding. Sidewall 34 is substantially cylindrical in shape having circular cross section, however other shapes could also be used. Top 12 features three knobs 14 which serve to enhance muscular or sexual stimulation by concentrating the pressure and motion of cap 10 to several discrete points on the user. Although any number and configuration of knobs can be used, three knobs placed in an equilateral triangle formation are believed to be particularly effective for the desired stimulation.

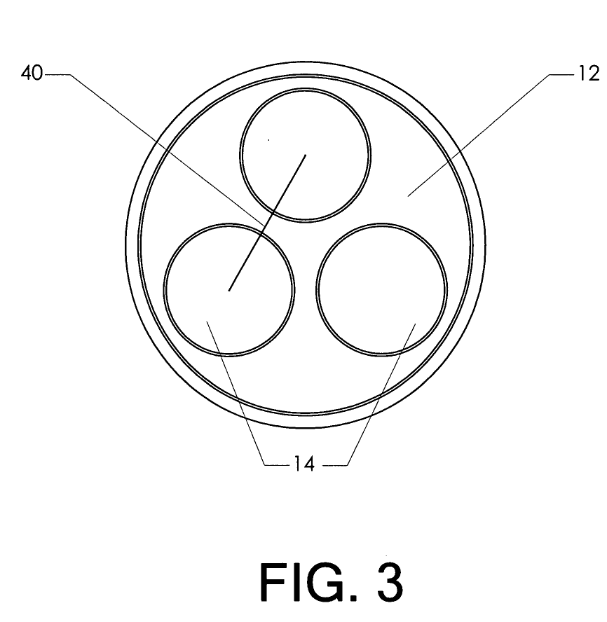

FIG. 3 shows the topographical layout of a preferred embodiment. Top 12 is shown with three knobs 14. Although cap 10 can be any size, the preferred range for the diameter is 1.5 cm to 3.0 cm. Knobs 14 are illustrates in FIG. 3 as half spheres although other shapes can also be used for knobs 14 such as cylinders, rounded cylinders, rectangular prisms, pyramids, and cones. Although knobs 14 can be any size, the preferred range for the diameter is 0.5 cm to 1.5 cm. An equilateral triangular formation with knob-to-knob distances 40 in the range of 0.5 cm to 1.5 cm is also preferred.

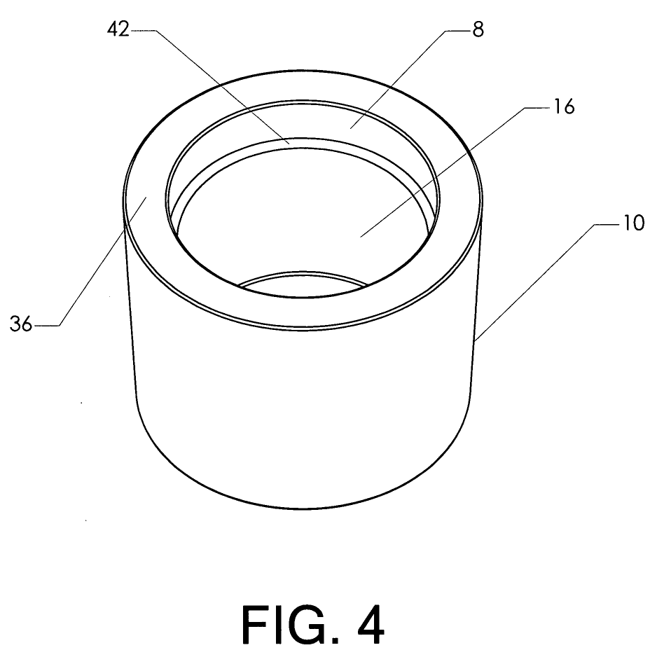

FIG. 4 shows the molded cap from the bottom. Bottom 36 of cap 10 has bore 16. In the preferred embodiment, bore 16 terminates near the top of cap 10. Bore 16 is preferably sized to receive bristles 32 of electric toothbrush 38 which is typically in the range of 1 cm to 1.5 cm. Counterbore 18 is also provided in bottom 36 of cap 10. The difference in diameter of counterbore 18 and bore 16 forms brush-head seat 42 which mates with brush head 30 of electric toothbrush 38 when bristles 32 are inserted into bore 16.

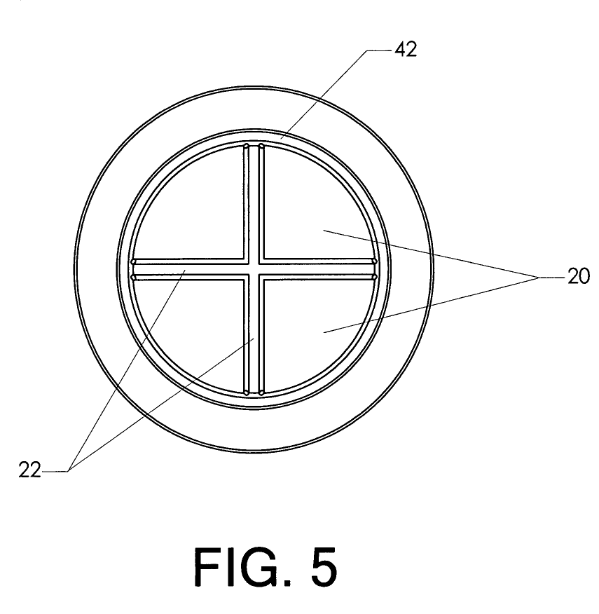

A bottom view of an alternate embodiment of the present invention is shown in FIG. 5. Cap 10 has two perpendicularly-situated ridges 22 in the bottom of bore 16. Ridges 22 create four compartments 20 in the bottom of bore 16. When bristles 32 are inserted into bore 16 and brush head 30 is mated with brush-head seat 42, bristles 32 separate into the four compartments 20. Ridges 22 provide a surface for bristles 32 to engage when electric toothbrush 30 is switched on, thereby transferring the motion of brush head 30 and bristles 32 to cap 10.

Motion of brush head 30 and bristles 32 can also be transferred to cap 10 by the use of an adhesive applied to bristles 32, brush head 30, bore 16, and/or counterbore 18. The adhesive works to permanently affix the components of cap 10 to brush head 30, thereby insuring transfer of motion between the two parts. Any adhesive known in the art can be used to attach the components including glues, epoxies, silicone-based adhesives, and acrylic resins.

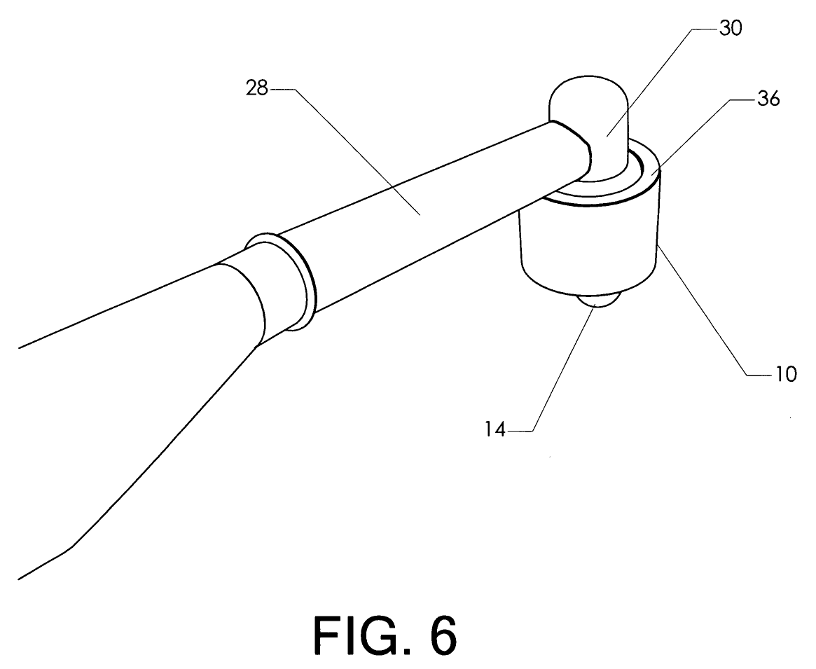

FIG. 6 shows the cap attached to the electric toothbrush. Cap 10 and electric toothbrush 38 are attached by the insertion of bristles 32 into bore 16 of cap 10. As illustrated in FIG. 6, knobs 14 face away from brush head 30. Cap 10 has counterbore 18 (not shown here) so that brush head 30 also fits inside of cap 10, and the back side of brush head 30 is relatively flush with bottom 36 of cap 10.

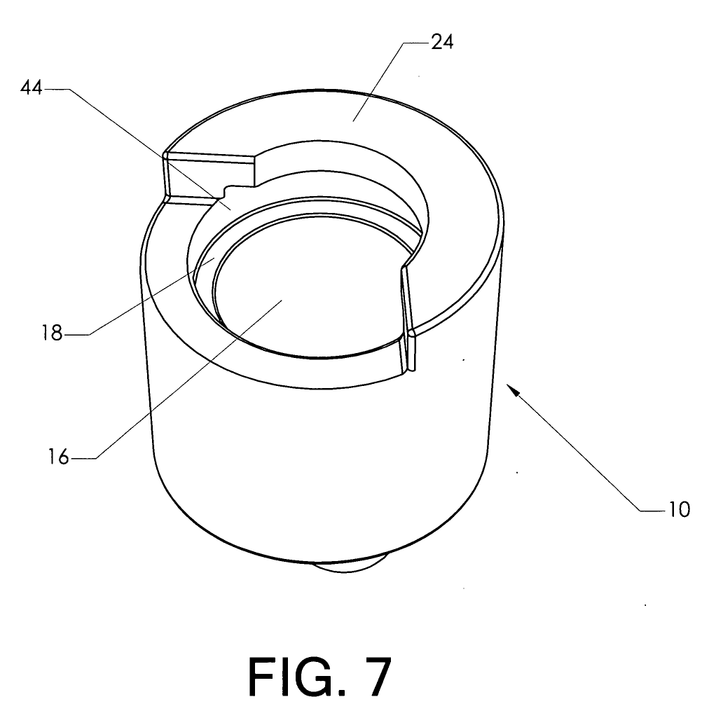

FIG. 7 shows an alternate embodiment of the cap. Cap 10 features counterbore 18 and partial-annular. lip 24. The addition of lip 24 creates groove 44 in the counterbored region beneath lip 24. Groove 44 helps prevent the accidental detachment of cap 10 from electric toothbrush 38 when the two are attached.

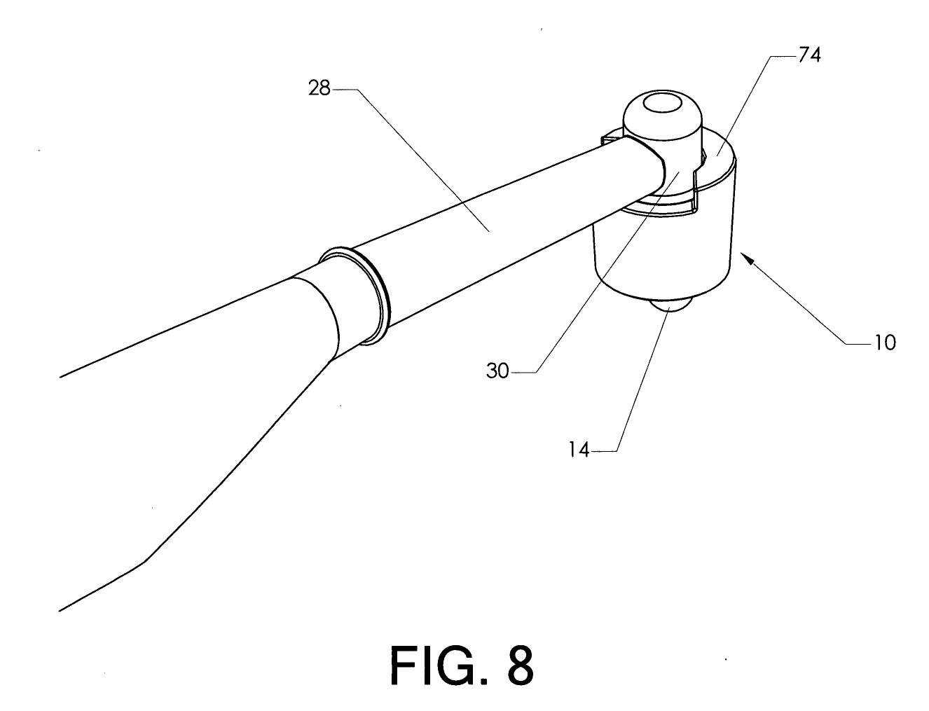

FIG. 8 shows the alternate embodiment of the cap attached to an electric toothbrush. Knobs 14 again face away from brush head 30. Bottom 36 is generally flush with the back side of brush head 30 except for lip 24 which overlaps brush head 30 thereby securing cap 10 to the brush.

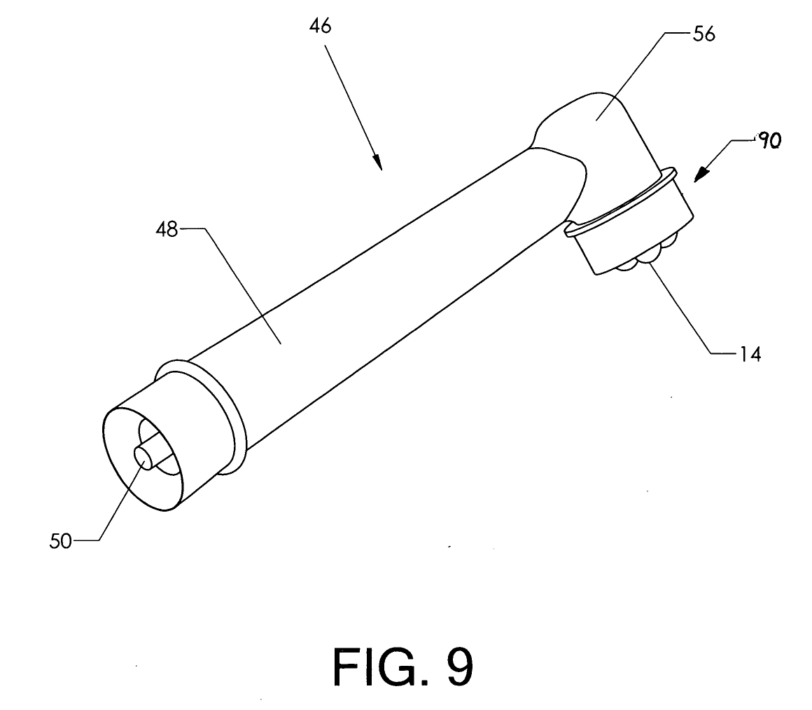

FIG. 9 shows another alternate embodiment of the present invention. In this embodiment tip member 90, wand 48, and head 56 are provided as a single, integrated unit, shaft assembly 46. In this embodiment, wand 48 and head 56 are substantially the same as the previously shown wand 28 and brush head 30 respectively. Wand 48, like the previous wand 28, contains a transfer (not shown) that converts the motion created by the internal motor of the previously shown electric toothbrush 38 into an oscillating motion in head 56. Tip member 90, which is connected to head 56, oscillates alternating between a positive and negative angle of rotation.

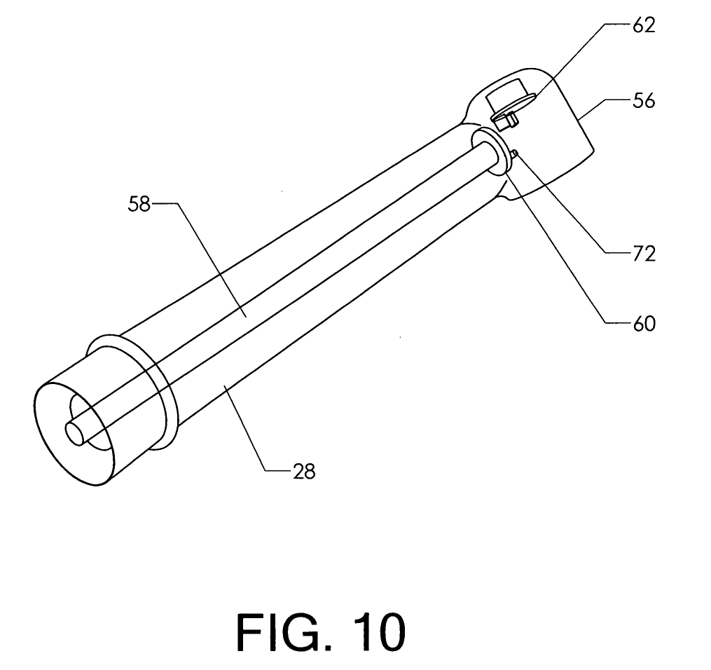

FIG. 10 shows the inside of wand 28 and head 56. Transfer 58 connects the internal motor of the previously shown electric toothbrush 38 to rotating disc 60. Rotating disc 60 rotates, drawing movement dowel 72, which is radially offset from the center of rotating disc 60, in a circle. Support disc 62 attaches directly to alternate head 56 and remains fixed in place.

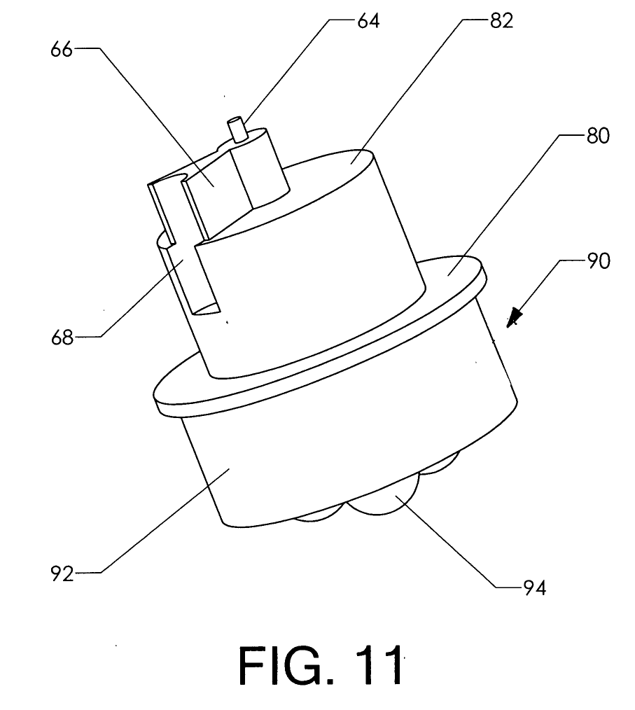

A perspective view of tip member 90 in the present embodiment is shown in FIG. 11. Back 80 of tip member 90 has an elevated edge 82. Support peg 64 sits in the center of back 80 and is directly attached to edge 82 of tip member 90. Two stop surfaces 66 extend radially from the center of back 80 where support peg 64 is located. The stop surfaces 66 define the degree of angular rotation. Slide 68 sits below stops 66 and has a hollow interior.

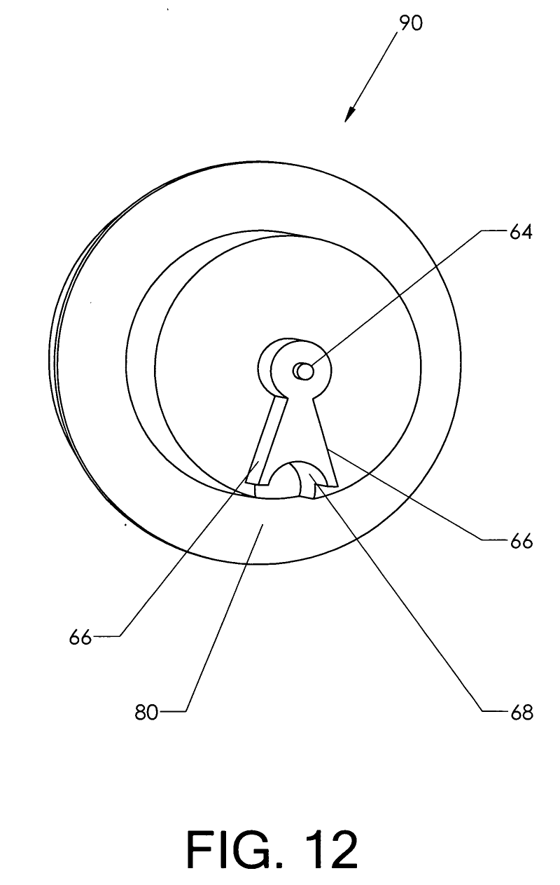

FIG. 12 shows back 80 of tip member 90 in the present embodiment. Both stop surfaces 66 can be seen from this view. Slide 68 has a cylindrical surface and is located in the angular separation between the two stop surfaces 66.

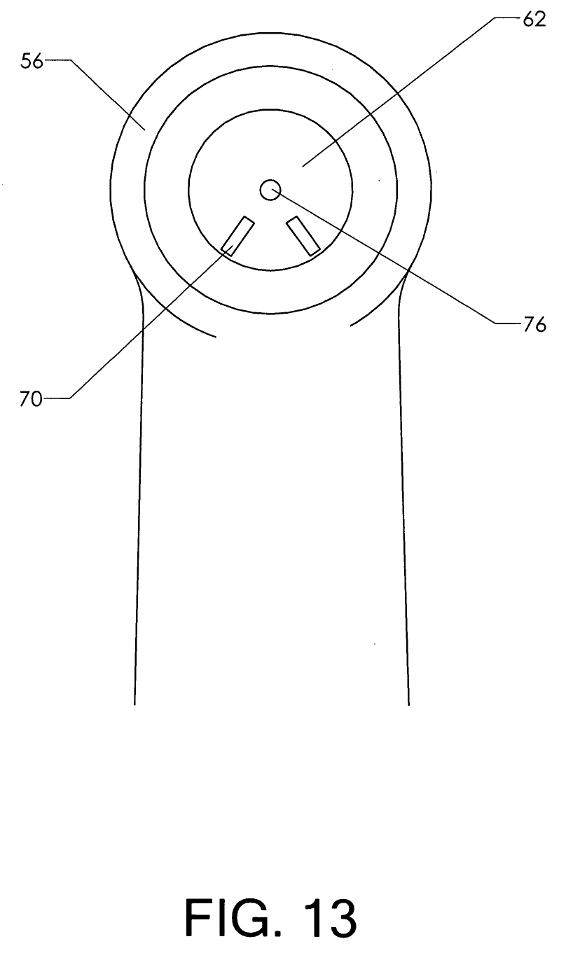

The front side of head 56 is shown in FIG. 13. Notch 76 is located in the center of head 56. Support disc 62 is held in a stationary position on head 56. Two flanges 70 extend in the radial direction away from the central notch 76, which is located in the center of support disc 62. The two flanges 70 are angularly separated by approximately 90 degrees.

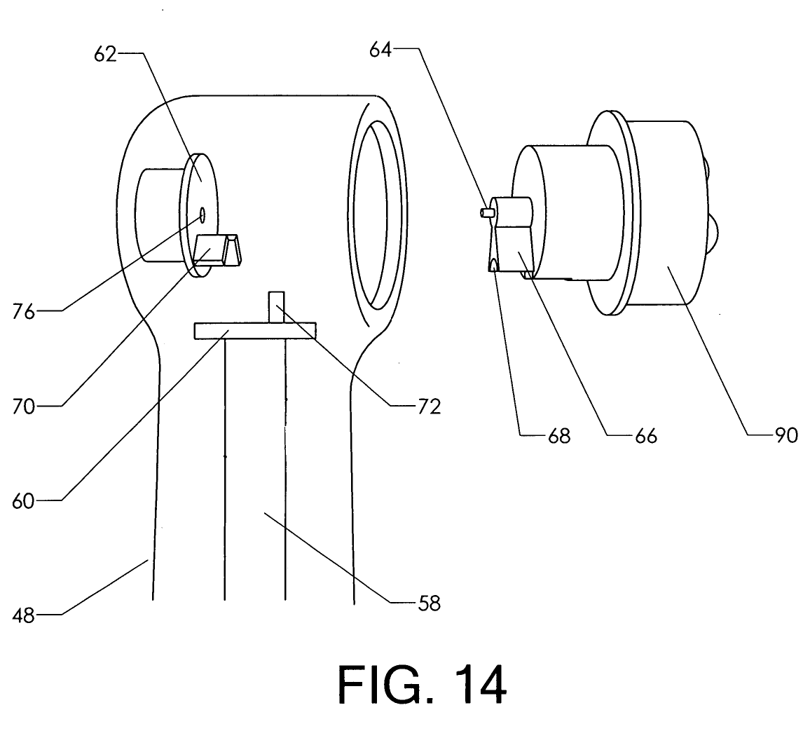

FIG. 14 shows the attachment of tip member 90 to alternate wand 48. Support peg 64 fits into notch 76 and movement dowel 72 fits into slide 68. As rotating disc 60 turns, movement dowel 72 is drawn in a circle around the perimeter of rotating disc 60. Movement dowel 72 pushes against the side wall of slide 68 as it circles to one side of rotating disc 60. The force exerted against the side wall of slide 68 forces tip member 90 to rotate in one direction. Flange 70 abuts against stop 66 when movement dowel 72 is at the farthest point to one side of rotating disc 60 thus stopping the rotational motion of tip member 90. As movement dowel 72 transitions to the other side of rotating disc 60 the process repeats itself in the opposite direction.

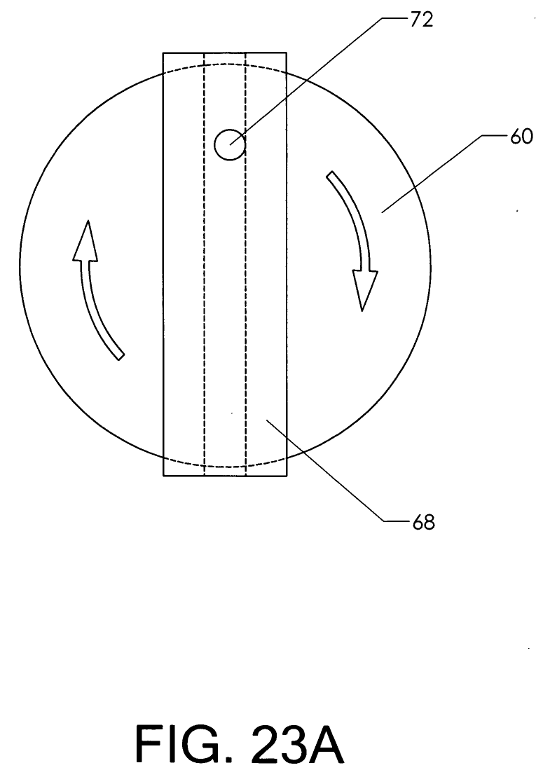

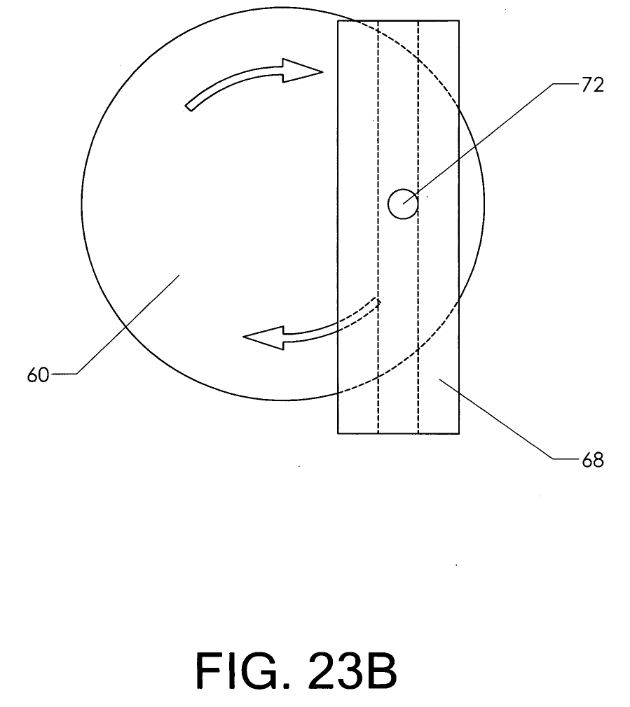

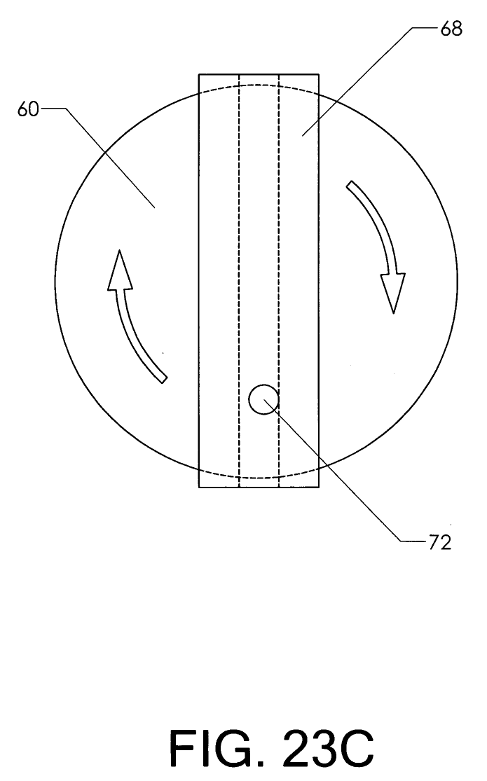

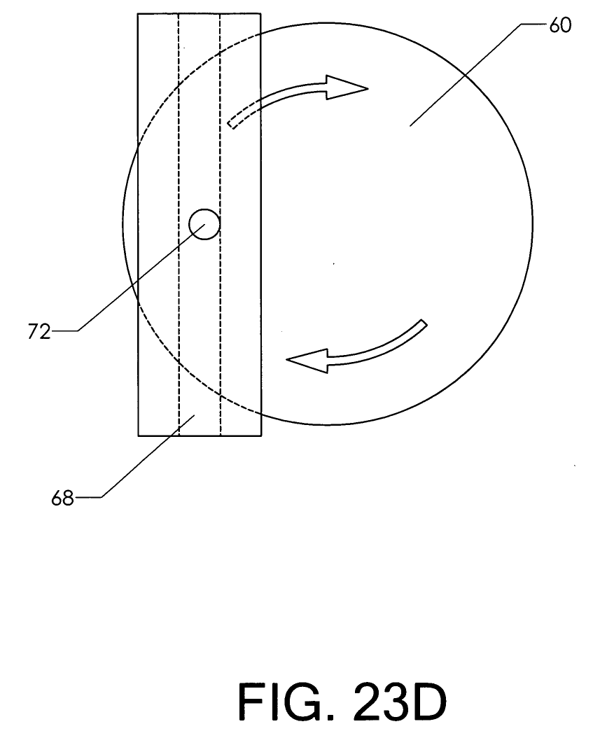

FIG. 23A-23D show the motion of movement dowel 72 relative to slide 68. FIG. 23A shows movement dowel 72 in position 1, a centrally located position with respect to the motion of slide 68. FIG. 23B shows movement dowel 72 in position 2. As rotating disc 60 draws movement dowel 72 in a circle, slide 68 is pushed to one side of rotating disc 60. As movement dowel 72 reaches position 2 slide 68 is forcing tip member 90 (not shown) into a positive angular rotation. In FIG. 23C movement dowel 72, shown in position 3, has again circled to a centrally located position of rotating disc 60 with respect to the motion of slide 68, however movement dowel 72 is now on the opposite side of rotating disc 60 as it was in position 1. As the reader will observe even though movement dowel 72 is in a different location than position 1, the slide is in substantially the same position as it was in position 1. Therefore, tip member 90 (not shown) is in substantially the same position when movement dowel 72 is in position 1 and position 3. As movement dowel 72 moves to position 4, shown in FIG. 23D, tip member 90 is forced into a negative angular rotation. Movement dowel 72 next circles back to position 1, shown in FIG. 23A, bringing slide 68 back to the center of rotating disc 60.

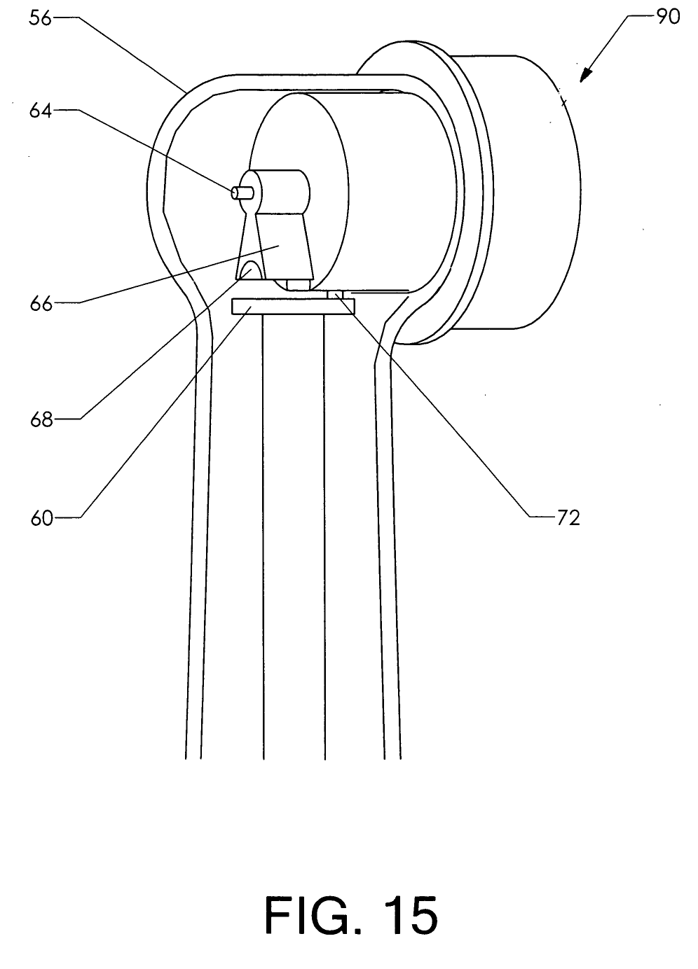

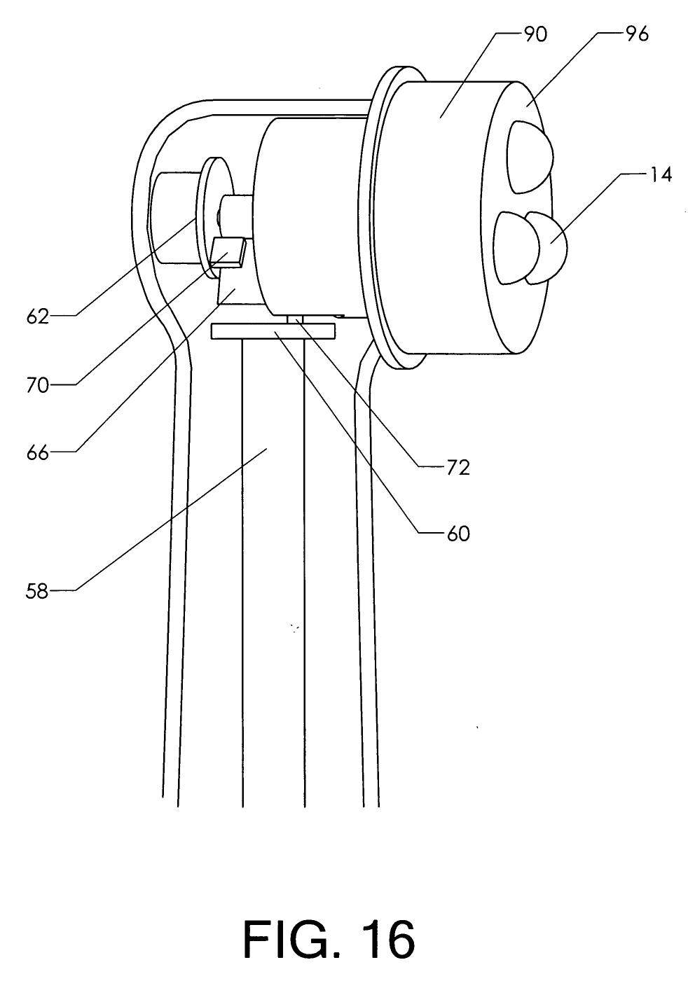

FIG. 15 shows how tip member 90 fits into head 56. Movement dowel 72 is shown in the furthest forward position as it fits into slide 68. The fully assembled and integrated device is shown in FIG. 16. Support disc 62 can be flush against the backside of tip member 90. As transfer 58 rotates, tip member 90 oscillates from one direction to the other.

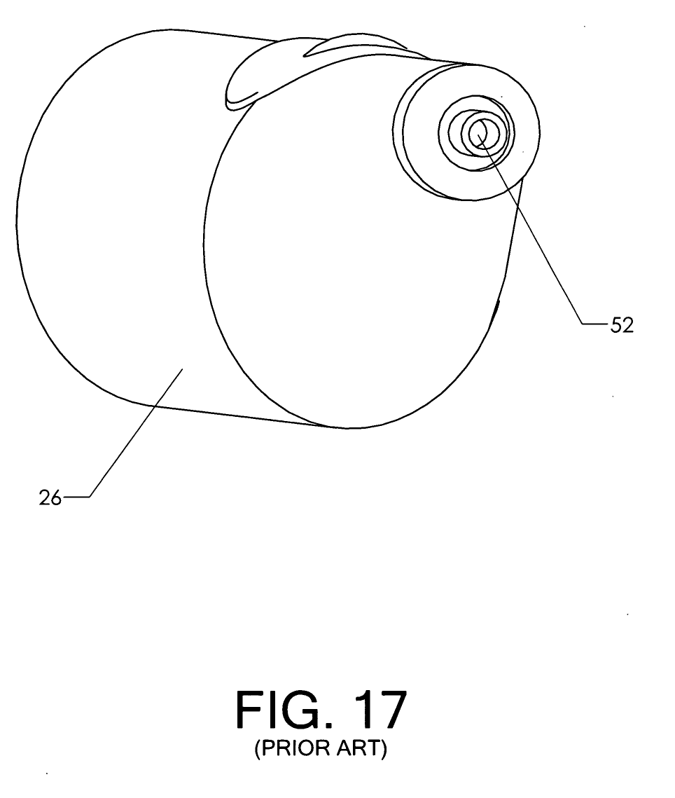

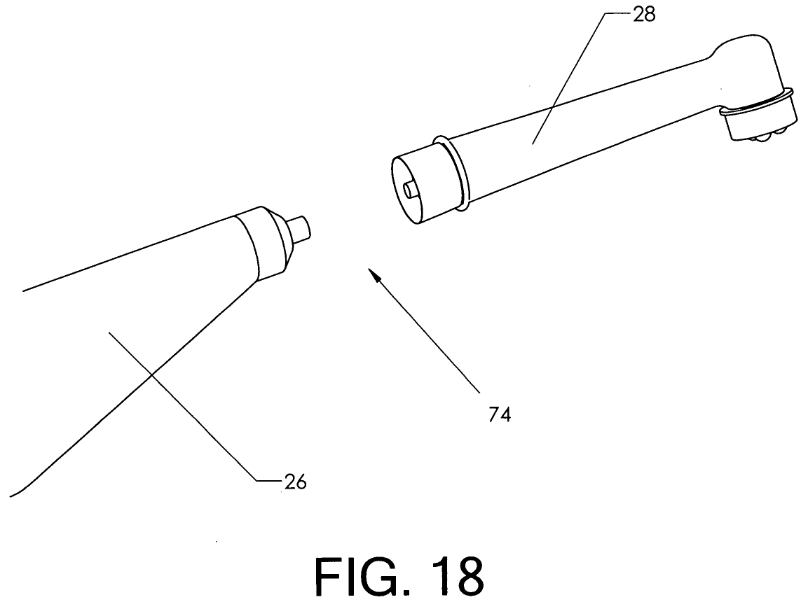

A perspective view of the prior art grip 26 with female coupling portion 52 is shown in FIG. 17. The motor (not shown) is housed inside of grip 26. The energy created in the motor is conveyed to wand 28 by a prior art coupling 74 between electric toothbrush 26 and wand 28 as shown in FIG. 18. The reader should appreciate that any manner of coupling the electric toothbrush 26 to wand 28 could be used to achieve the same results.

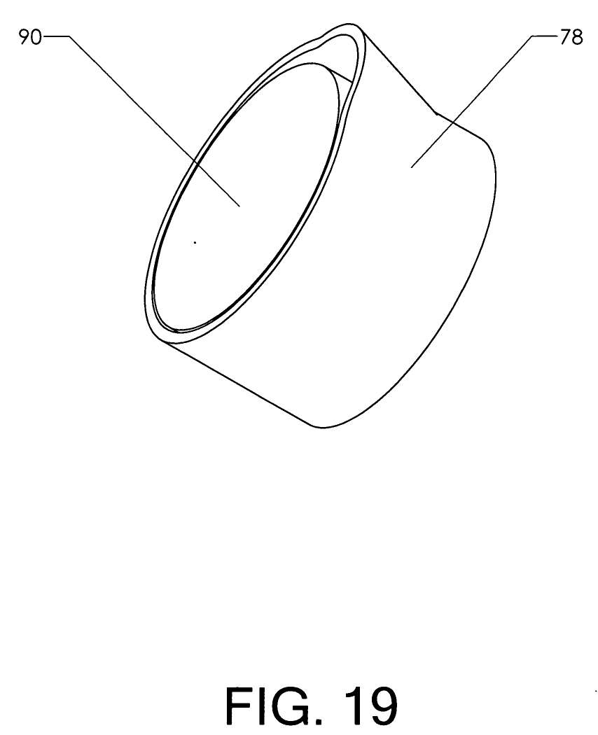

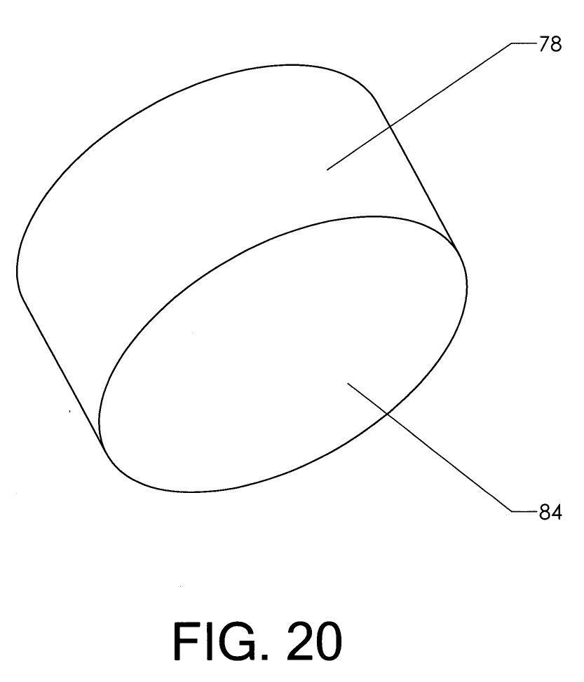

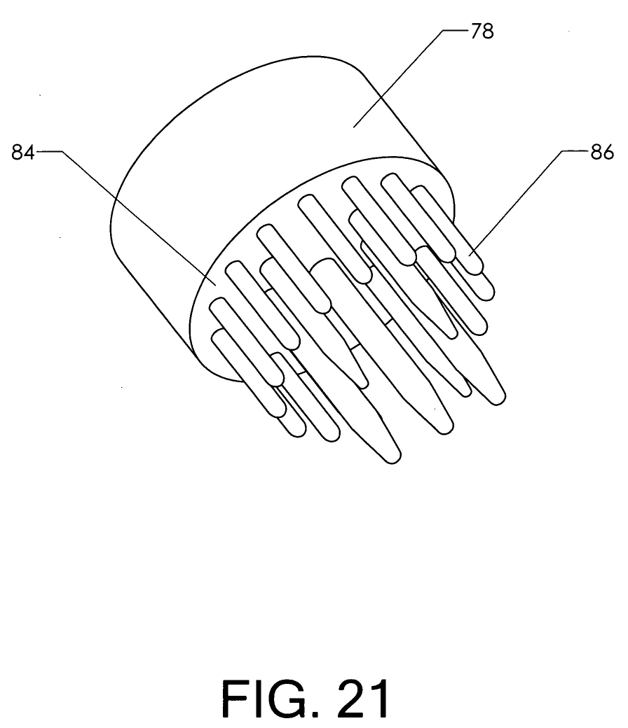

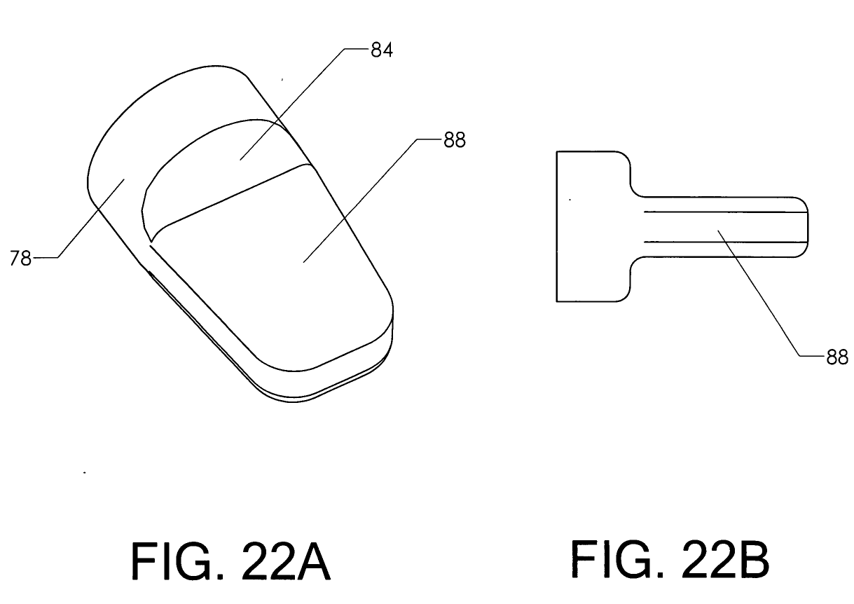

In yet another embodiment of the present invention it is desirable to have a deformable cap 78, shown in FIG. 19, which fits over tip member 90. In order to illustrate the ability of deformable cap 78 to stretch, a portion of deformable cap 78 is shown pulled away from tip member 90. Deformable cap 78 frictionally engages tip member 90 in order to allow the user to experiment with protrusions of different shapes and texture. FIG. 20 shows deformable cap 78 including crown 84. The protrusions are located on crown 84 of deformable cap 78. For example, FIG. 21, shows a multitude of fingers 86 protruding from crown 84 of deformable cap 78. Additionally, FIG. 22 shows two views of a single flattened ledge 88 protruding from crown 84 of deformable cap 78. In the preferred embodiment deformable cap 78 is made of material which is pliable, such as rubber or a deformable plastic.

The preceding description contains significant detail regarding the novel aspects of the present invention. It is should not be construed, however, as limiting the scope of the invention but rather as providing illustrations of the preferred embodiments of the invention. As an example, variations in the number, shape, or configuration of the knob-like protrusions can be employed without altering the function of the invention. Thus, the scope of the invention should be fixed by the following claims, rather than by the examples given.

Having described my invention, I claim:

1. An attachment for an electric toothbrush for providing stimulation to a portion of skin of a user, said electric toothbrush having a motor and a grip, said attachment comprising:
a. a wand having a first end and a second end, said first end of said wand attachable to said grip of said electric toothbrush, said second end terminating in a head, said wand having a hollow interior and shaft extending between said first end and said second end of said wand through said hollow interior.
b. a tip member, said tip member having a skin contacting surface, a back portion, and a sidewall extending therebetween, said tip member attachable to said head of said wand at said back portion of said tip member;
c. wherein when said tip member is attached to said head of said wand, said tip member is mechanically linked to said shaft and is configured to move relative to said head when said attachment is attached to said electric toothbrush and said motor is activated.

2. The attachment of claim 1, wherein said tip member is configured to provide sexual stimulation to said portion of skin of said user.

3. The attachment of claim 1, wherein said tip member rotates angularly with respect to said head.

4. The attachment of claim 3, wherein said tip member oscillates between a positive and a negative angular rotation.

5. An attachment of claim 1, wherein said tip member is configured to provide a contact surface allowing said tip member direct contact with said portion of skin of said user.

6. The attachment of claim 1, wherein said top portion of said tip member has a knob-like protrusion.

7. The attachment of claim 1, wherein said shaft further comprises a rotating disc, having a center and a perimeter, attached to said shaft, proximal said head, wherein said rotating disc further comprises a movement dowel, wherein said movement dowel is radially offset from said center of said rotating disc.

8. The attachment of claim 7, wherein said shaft transmits the motion of said motor from said electric toothbrush to said tip member.

9. The attachment of claim 1, wherein said attachment further comprises a deformable cap, said deformable cap having a crown.

10. An attachment for an electric toothbrush for providing stimulation to a portion of skin of a user, said electric toothbrush having a motor and a grip, said attachment comprising:
a. a wand having a first end and a second end, said first end of said wand attachable to said grip of said electric toothbrush, said second end terminating in a head, said wand having a hollow interior and shaft extending between said first end and said second end of said wand through said hollow interior.
b. a tip member, said tip member having a skin contacting surface, a back portion, and a sidewall extending therebetween, said tip member attachable to said head of said wand at said back portion of said tip member; and
c. a deformable cap, said deformable cap having a crown;
d. wherein when said tip member is attached to said head of said wand, said tip member is mechanically linked to said shaft and is configured to move relative to said head when said attachment is attached to said electric toothbrush and said motor is activated.
e. wherein said deformable cap is comprised of a pliable material, said pliable material having the ability to stretch over and engage said tip member.

11. An attachment of claim 10, wherein said deformable cap is configured to provide a contact surface allowing said deformable cap direct contact with said portion of skin of said user.

12. At attachment of claim 10, wherein said tip member is configured to rotate angularly with respect to said head.

13. An attachment of claim 10, wherein said crown of said deformable cap further comprises a plurality of protrusions.

14. An attachment of claim 10, wherein said crown of said deformable cap further comprises at least one protrusion.

15. The attachment of claim 12, wherein said tip member oscillates between a positive and a negative angular rotation.

16. The attachment of claim 10, wherein said deformable cap oscillates between a positive and a negative angular rotation.

17. The attachment of claim 10, wherein said crown of said deformable cap is smooth.

18. The attachment of claim 10, wherein said shaft further comprises a rotating disc, having a center and a perimeter, attached to said shaft, proximal said head, wherein said rotating disc further comprises a movement dowel, wherein said movement dowel is radially offset from said center of said rotating disc.

19. The attachment of claim 18, wherein said shaft transmits the motion of said motor from said electric toothbrush to said tip member.
